(19) United States
(12) Patent Application Publication
(10) Pub. No.: US 2017/0258984 A1
(43) Pub. Date: Sep. 14, 2017

(54) MEDICAL BEDSIDE SAFETY MODULAR APPARATUS AND METHOD

(71) Applicant: Variety Children's Hospital d/b/a Nicklaus Children's Hospital, Miami, FL (US)

(72) Inventor: George Keith Meyer, Miami, FL (US)

(21) Appl. No.: 15/067,722

(22) Filed: Mar. 11, 2016

(51) Int. Cl. A61M 5/14; F16L 3/08; F16L 3/22

(52) U.S. Cl. CPC A61M 5/1418; F16L 3/222; F16L 3/08; A61M 2205/60

(57) ABSTRACT

A medical line organizer that can include multiple attachable guides for separating and visually distinguishing between attached medical line guides is provided. The guides can be attached to each other or to one or more couplers for attachment to a structure. Various identification techniques can be used to distinguish the guides from each other for identification of individual lines. Additional guides or markers with similar indicia can be placed on the lines to aid in identifying the path of a line within a particular guide.

### BACKGROUND OF INVENTION

Patients in healthcare settings, in home or in a medical facility, are often in need of intravenous support for variable lengths of time. Such support is often provided in the form of catheterization of the patient, wherein a small diameter catheter is inserted into a vein or artery. The catheter is attached to one or more intravenous lines that lead to an infusion device of substances to be delivered directly into the patient's bloodstream. The intravenous lines typically attached to a catheter are flexible, usually non-opaque, tubing of a non-reactive material having a diameter sufficient to transport fluids, antibiotics, therapies, nutrition, and other substances or combinations thereof from the infusion device to the catheter. Depending on the acuity of the patient, the requirements for intravenous support may increase significantly, necessitating the use of multiple intravenous lines. It is not unusual in some cases for a patient to have several intravenous lines originating from multiple infusion devices. As the need for more intravenous lines increases, so does the complexity of the environment around a patient. This increasing complexity can further create a potentially unsafe situation for the patient and for the healthcare staff, who can trip or become entwined in the lines.

The simultaneous use of several intravenous lines also creates difficulty in quick, emergent identification of a particular medication or fluid being delivered to a patient. The visual complexity of multiple similar-looking intravenous lines can be difficult to sort out and can lead to dangerous errors. For example, if a patient is receiving multiple continuous infusions and/or medications, a situation may arise wherein the healthcare provider is required to identify and discontinue one of the infusion processes. Multiple, unidentified, identical lines, can create a delay in care and a safety issue for the patient, as the healthcare provider sorts out the lines to find the correct one to close or remove.

A further issue is the potential for tangled lines. This can be particularly problematic with pediatric patients, who tend to move more than adult patients, and during patient transport. Intravenous lines are often transported with patents. As a result, healthcare providers can spend valuable time untangling intravenous lines whenever a patient arrives at a new destination.

There have been a number of devices that have been developed to try to address these issues. Unfortunately, most of them are single unit devices that difficult for healthcare providers to set up quickly and manage effectively.

### BRIEF SUMMARY

The embodiments of the subject invention relate, in general, to devices and methods for organizing and securing medical lines, such as medical tubing and similar types of apparatuses. More specifically, embodiments of the subject invention pertain to a modular medical line organizer for arranging and supporting such apparatuses as medical tubing, intravenous (IV) lines, electrical connections, and other elongate, flexible, apparatuses typically present in medical bedside environments. The device and methods disclosed herein successfully address the disadvantages that have been associated with the previously known devices and methods, and provides certain attributes and advantages, which have not been realized by these known devices. In particular, the embodiments of the subject invention provide novel, inexpensive, and highly effective methods and devices for convenient and effective organization, labeling, and maintenance of medical lines and tubing.

Embodiments of the subject invention provide a modular device incorporating one or more individually identifiable guides. One or more medical lines, particularly IV lines, or other elongate, flexible apparatuses typically in a patient bedside environment can be inserted through a guide and into a lumen of the guide. The shape of the lumen can hold the guide onto the medical line. Guides can be lightweight and float freely on the medical lines and provide for ease of identification of individual lines and tubing. Advantageously, the guides can further have interlocking tracks that allow them to be joined together for holding multiple lines in an organized structure. A further advantageous feature is the ability of the guides to be attached to one or more couplers that can secure the one or more joined guides to a bedside rail, IV stand, neonatal isolette, or other bedside support.

Additional embodiments can include markers associated with each identifiable guide, where one or more similarly identifiable markers can be placed along the length of a medical line secured within a similarly identifiable guide. When a guide is attached to a bedside support, the associated, similarly-identifiable markers, placed on the medical line, allow for easier identification of that particular line as it traverses through the patient environment. The markers can also have interlocking tracks that allow them to be attached to their respective guides for organization of the guides and markers when one or both are not in use.

The modular system embodiments of the subject invention can be easy to set up and maintain and can increase efficiency and safety in a patient environment. They can be easy to use and are capable of being interchanged as necessary during initial setup or while in use. Guides and markers are amenable to a variety of identification systems based on color, number, symbol, shape, or other systems, or combinations thereof. Specific embodiments are disposable, cost effective to implement in practically any medical setting, do not require alteration of existing equipment, and do not affect the operation or functionality of any apparatus with which they are engaged.

It should be noted that this Brief Summary is provided to generally introduce the reader to one or more select concepts described below in the Detailed Disclosure in a simplified form. This Summary is not intended to identify key and/or required features of the claimed subject matter. Other aspects and further scope of applicability of the present invention will also become apparent from the detailed descriptions given herein. It should be understood, however, that the detailed descriptions, while indicating preferred embodiments of the invention, are given by way of illustration only, since various changes and modifications within the spirit and scope of the invention will become apparent from such descriptions. The invention is defined by the claims below.

### BRIEF DESCRIPTION OF DRAWINGS

In order that a more precise understanding of the above recited invention can be obtained, a more particular description of the invention briefly described above will be rendered by reference to specific embodiments thereof that are illustrated in the appended drawings. The drawings presented herein may not be drawn to scale and any reference to dimensions in the drawings or the following description is specific to the embodiments disclosed. Any variations of these dimensions that will allow the subject invention to function for its intended purpose are considered to be within the scope of the subject invention. Thus, understanding that these drawings depict only typical embodiments of the invention and are not therefore to be considered as limiting in scope, the invention will be described and explained with additional specificity and detail through the use of the accompanying drawings in which:

FIG. 1 is an illustration of one embodiment of a medical line organizer, according to the subject invention.

FIG. 2 is a rear face view of the medical line organizer shown in FIG. 1.

FIG. 3A is a front face view of one embodiment of a guide, according to the subject invention.

FIG. 3B is a photograph of a front face view of an embodiment of a guide, according to the subject invention.

FIG. 4 is a front face view illustrating an embodiment where two guides having male and female tracks are sliding together.

FIG. 5 is a distal end view of a guide and two partial guides that shows one embodiment of male tracks and female tracks, according to the subject invention.

FIG. 6A is an illustration of a cross-sectional view taken along line A-A′ in FIG. 6B. This view illustrates one embodiment of a track stop utilizing wings on the male track.

FIG. 6B is a photograph of the proximal end of a male track, according to the subject invention, which illustrates a wing embodiment of a track stop.

FIG. 7 is an illustration of three guides operatively connected according to embodiments of the subject invention.

FIG. 8A is a photograph of one embodiment of a coupler and an attached holder, according to the subject invention. This view shows one embodiment of a female track of the subject invention.

FIG. 8B is a photograph of a side elevation of the coupler and attached holder shown in FIG. 8A. This view shows one embodiment of a male track of the subject invention.

FIG. 9 is a photograph of a medical line organizer with color and alphanumeric indicia.

FIG. 10 is a photograph of one embodiment of a guide and one embodiment of a marker that are removably attached, according to the subject invention.

FIG. 11 is a photograph that illustrates how embodiments of a guide and a marker can be attached to a single medical line, according to the subject invention.

FIG. 12 is a photograph showing a proximal end of a medical line organizer, according to the subject invention.

FIG. 13A is an illustration of an embodiment of a medical line organizer, according to the subject invention, shown attached to a bedside structure. The couplers shown in this embodiment have both male and female tracks.

FIG. 13B is an illustration of an embodiment of a medical line organizer having couplers with either male or female tracks.

FIG. 14 is an illustration of a rear face view of a medical line organizer utilizing removable holders. In this embodiment, a flexible band is shown attached through a sleeve to the coupler.

### DETAILED DISCLOSURE

The subject invention describes embodiments of an apparatus for organizing and securing medical lines or medical tubing. More specifically, the subject invention provides one or more embodiment(s) of a modular medical line organizer of attachable line guides capable of sorting, identifying, and securing multiple lines of medical tubing, electrical connectors, or similar such elongate, flexible apparatuses often found at a patent bedside or in the patient environment. The medical line organizer embodiments of the subject invention can be used effectively with a single medical line; but, they are advantageously amenable to being expanded as necessary, by the addition of more guides, to accommodate more than one line for any particular patient. Embodiments can be easily incorporated into the patient environment, such that, as the complexity of tubing within a patient environment increases and becomes more burdensome to sort and maintain, a tubing organizer can be introduced at any time to help maintain organization of the multiple lines. Further embodiments include line markers that can be associated with a particular guide and attached along the length of a medical line to aid identifying the path of a medical line secured within a particular guide. Additionally, one or more couplers can be used to attach and/or secure one or more guides to a bedside structure, such as, for example, a bed rail, IV stand, medical device or equipment, or, in some embodiments, directly to a patient.

The following description will disclose that the subject invention is particularly useful in medical environments where a patient may be connected to multiple medical devices, in particular one or more IV tubings or medical device connectors. However, a person with skill in the art will be able to recognize numerous other uses that would be applicable to the devices and methods of the subject invention. While the subject application describes, and many of the terms herein relate to, a use for organizing IV tubing, other uses and modifications apparent to a person with skill in the art and having benefit of the subject disclosure are contemplated to be within the scope of the present invention.

In the description that follows, a number of terms used relate to treatment of a patient in a medical or pseudo-medical environment. In order to provide a clear and consistent understanding of the specification and claims, including the scope to be given such terms, the following definitions are provided.

As used herein, the term “patient” is used to refer to any vertebrate species, such as humans and animals. Preferably, the patient is of a mammalian species. Mammalian species which benefit from the disclosed methods of treatment include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Human or non-human animal patients can range in age from neonates to elderly.

The term “healthcare provider” as used in the subject invention is merely for literary convenience. The term should not be construed as limiting in any way. The devices, apparatuses, methods, techniques and/or procedures of the subject invention could be utilized by any person desiring or needing to do so and having the necessary skill and understanding of the invention.

As used herein, the phrase “patient environment” refers to the area around a patient, whether in a home, medical facility, or other environment, that a healthcare provider may occupy when tending to the patient or where medical equipment, medical machines, or any other apparatus associated with care of the patient may be located. Thus, it will be understood that the radius of a patient environment can vary.

As used herein, the phrase “medical line” refers to any elongated, generally flexible, apparatus used to operably connect a patient to another device. This can include, but is not limited to, different types of tubing for use with various instruments or machines or for specific uses, e.g., fluid management, drainage, anesthesia, respiration, intravenous (IV), and pumps. It can also include electrical connectors and wires used to operably connect a patient to any one or more of a variety of medical devices or equipment, e.g., monitors, scanners, biofeedback devices, stimulators, regulators, and alarms. Thus, it will be understood by a person skilled in the art that the embodiments of the subject invention can be utilized with any type of apparatus that operably connects a patient to a device or object, a device to another device, any line, attached or unattached, within a patient environment.

Any reference in this specification to “one embodiment,” “an embodiment,” “example embodiment,” “further embodiment,” “alternative embodiment,” etc., is for literary convenience. The implication is that any particular feature, structure, or characteristic described in connection with such an embodiment is included in at least one embodiment of the invention. The appearance of such phrases in various places in the specification does not necessarily refer to the same embodiment. Further, when a particular feature, structure, or characteristic is described in connection with any embodiment, it is within the purview of one skilled in the art to affect such feature, structure, or characteristic in connection with other ones of the embodiments.

Also, as used herein, and unless otherwise specifically stated, the terms “operable communication,” “operable connection,” “operably connected,” “cooperatively engaged” and grammatical variations thereof mean that the particular elements are connected in such a way that they cooperate to achieve their intended function or functions. The “connection” or “engagement” may be direct, or indirect, physical or remote.

Finally, reference is made throughout the application to the “distal end” and the “proximal end.” As used herein, the distal end is that end which has an opening for receiving an object therethrough. Conversely, the proximal end of the device is that end which is opposite to the distal end and, further, is closed or prevents an object from passing through.

The present invention is more particularly described in the following examples that are intended to be illustrative only since numerous modifications and variations therein will be apparent to those skilled in the art. As used in the specification and in the claims, the singular for “a,” “an” and “the” include plural referents unless the context clearly dictates otherwise.

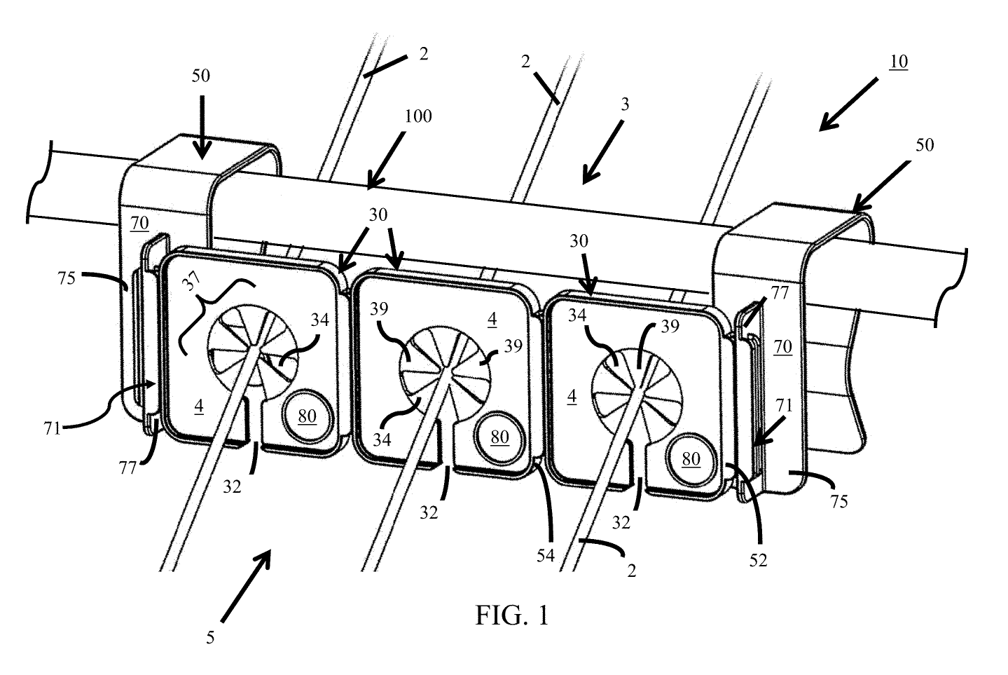

Reference will be made to the attached figures on which the same reference numerals are used throughout to indicate the same or similar components. With reference to the attached figures, which show certain embodiments of the subject invention, it can be seen in FIG. 1 that a medical line organizer 10, of the subject invention, generally comprises one or more interlocking guides 30 having an insertion slot 32 that leads to a central orifice 34 with a diaphragm 37 therein designed to maintain a medical line 2 within the orifice. Two or more guides 30 can be interlocked with the use of connectable structures 40, which can also allow the guides to be interlocked with one or more couplers 70 for securing the one or more guides to a bedside support 100. Additional embodiments can include one or more indicium 80 that distinguish the one or more guides or couplers 70 and at least one marker 90 associated with each guide.

In one embodiment, a guide 30 is a rigid or semi-rigid panel with a front face 4 and a rear face 6. The circumferential edge 8 of a guide can assume any polygonal shape that allows one or more guides to be connectable, as will be described below. FIGS. 1, 3A and 10 show embodiments where the circumferential edge is in the shape of a rounded-corner square. Other embodiments can include, but are not limited to, circumferential edge shapes that are round, triangular, rectangular, or other polygonal shapes. As will be discussed below, embodiments of the guide can be connected to each other and to one or more couplers 70. Accordingly, shape of a guide can be determined by the type of connectable structures 40 employed with the guides. A person with skill in the art can determine an appropriate shape for circumferential edge 8, according to the embodiments described herein. Such variations are within the scope of this invention.

The dimensions of a guide 30 can depend upon the shape employed for the circumferential edge 8. It can be beneficial for a guide to be sufficiently large enough that it can be easily identified or any one or more indicium 80 thereon can be accurately identified. It is expected that a guide will be located close to a patient, so as to be capable of being utilized with medical lines attached to the patient. It can be further beneficial if the dimensions of a guide do not cause the guide to inhibit the actions of a healthcare provider or interfere with any equipment in the patient environment. In one embodiment, a guide has a length (L) of at least 0.5, 1.0, 1.5, 2.0, 2.5, or 3.0 inches or a length in a range between any two of the listed values. In a further embodiment, a guide has a height (H) that is at least 0.5, 1.0, 1.5, 2.0, 2.5, or 3.0 inches or a height in a range between any two of the listed values. In a particular embodiment, a guide has a length (L) that is between approximately 1.5 inches to approximately 2.5 inches and a height (H) that is between approximately 1.5 inches and approximately 2.5 inches.

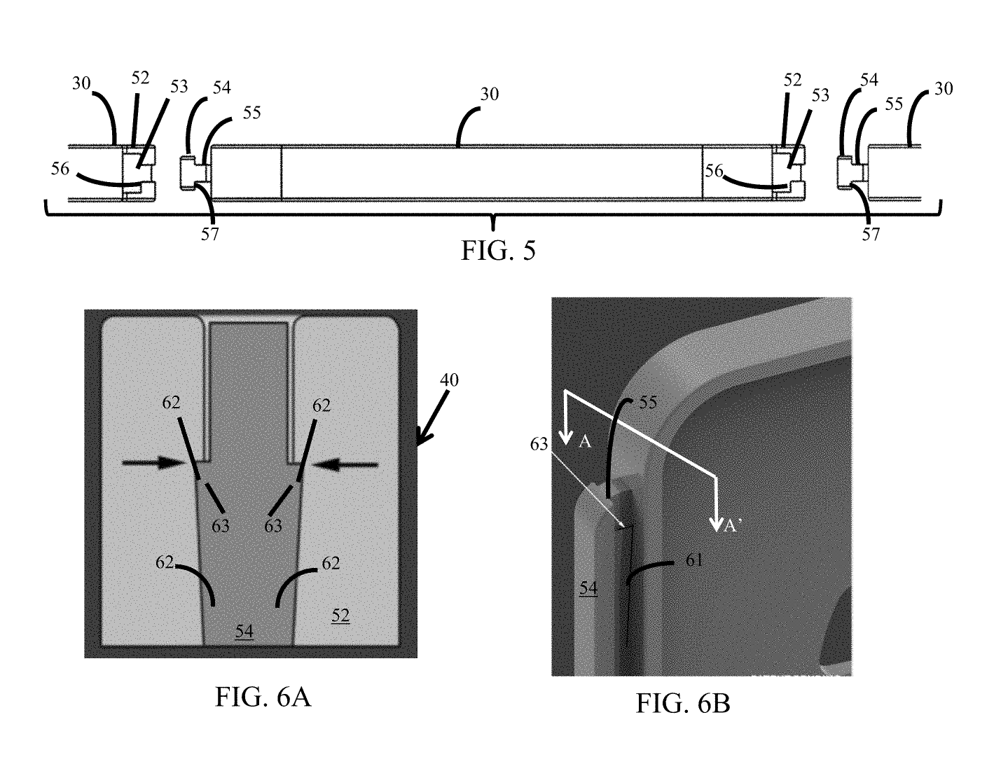

The width (W) of a guide can also vary and can depend upon multiple factors understood by those with skill in the art. In one embodiment, the width of a guide is at least 0.1, 0.15, 0.2, 0.25, 0.3, 0.35, 0.4, 0.45, 0.5, 0.55, and 0.6 inches or a width in a range between any two of the listed values. In a particular embodiment, the width of a guide is between approximately 0.3 inches and approximately 0.5 inches. FIGS. 5, 9, 10, and 11 illustrate a specific embodiment where a guide has a (L) and a (H) of approximately 2 inches and a maximum width of approximately 0.36 inches. Alternative combinations of dimensions are also possible. Such variations which provide the same function, in substantially the same way with substantially the same result are within the scope of this invention.

The material of a guide can be rigid or semi-rigid or some combination thereof. As will be discussed below, there are certain embodiments where it can be advantageous for at least a portion of the guide to be semi-flexible. Other embodiments to be discussed can incorporate various indicia 80 incorporated into a guide. In addition, the guides can be reusable or disposable. Thus, the selection of a material for a guide will depend upon these and other factors known to those with skill in the art. In a specific embodiment, a guide is made of a plastic or nylon material that imparts sufficient rigidity where necessary in some areas of a guide and sufficient flexibility where required in other areas of a guide.

For one or more medical lines to be organized and identifiable, they can be associated with a specific guide that, as will be discussed below, aids in identification of that specific medical line. This can entail a medical line being removably affixed to at least one guide. Ideally, removably affixing a medical line 2 to a guide 30 does not in any way inhibit the operation of the medical line or any materials, objects, electrical current, or other substance moving therethrough. In one embodiment, a guide has at least one orifice 34 therethrough, such that the front face 4 and the rear face 6 are communicated through the orifice. FIGS. 1, 2, 9 and 13A illustrate examples of an orifice. In these figures, the orifice is shown in a central location within a guide. It will be understood by a person skilled in the art that an orifice can be located anywhere on a guide and that more than one orifice can be present on a guide.

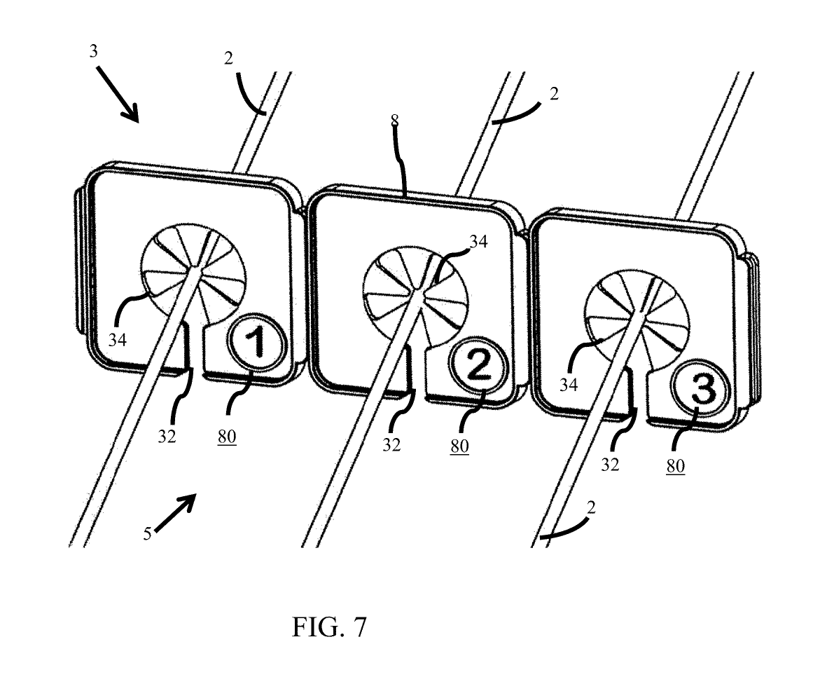

Access to the orifice can be achieved with an insertion slot 32 that extends through the circumferential edge 8 to the orifice, forming a channel within the guide that, like the orifice, communicates the front face 4 with the rear face 6. This can permit a medical line to be inserted, through the insertion slot so that it is arranged perpendicular to the front face 4 and rear face 6, examples of which are shown in FIGS. 7, 11, and 14. Typically, there is only one insertion slot 32 in a guide. The single insertion slot can provide access to a single orifice 34.

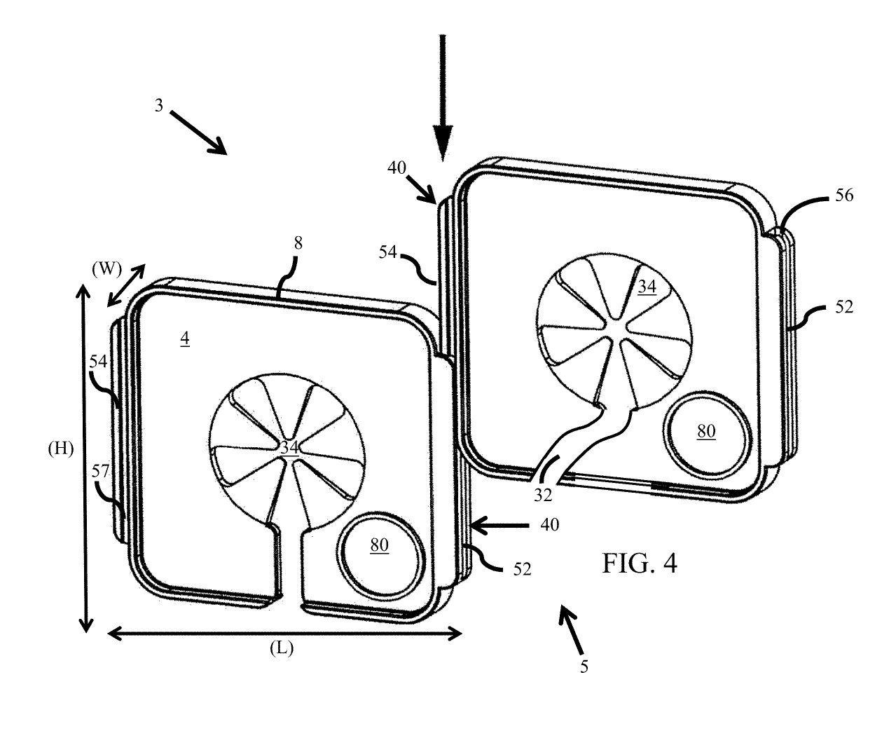

Typically, an insertion slot 32 is generally a straight channel from the circumferential edge 8 to the orifice 34. However, an insertion slot can assume any of a variety of paths or shapes, such that it does not have to follow a straight-line path. FIG. 4 illustrates an example where the insertion slot in a guide is curved.

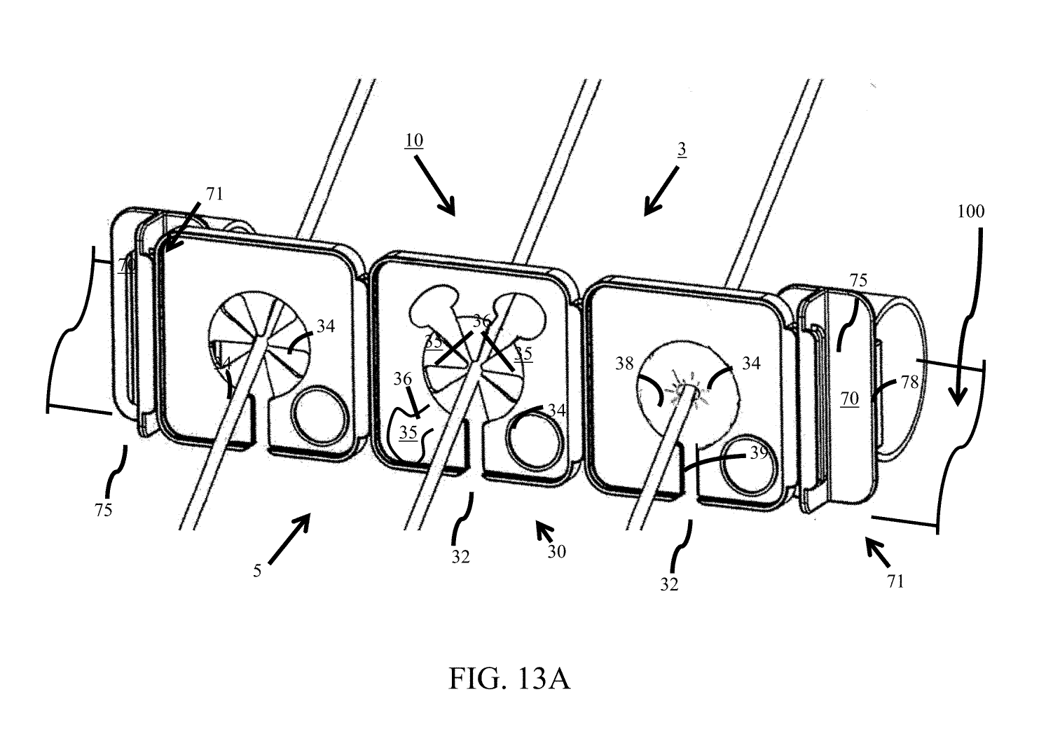

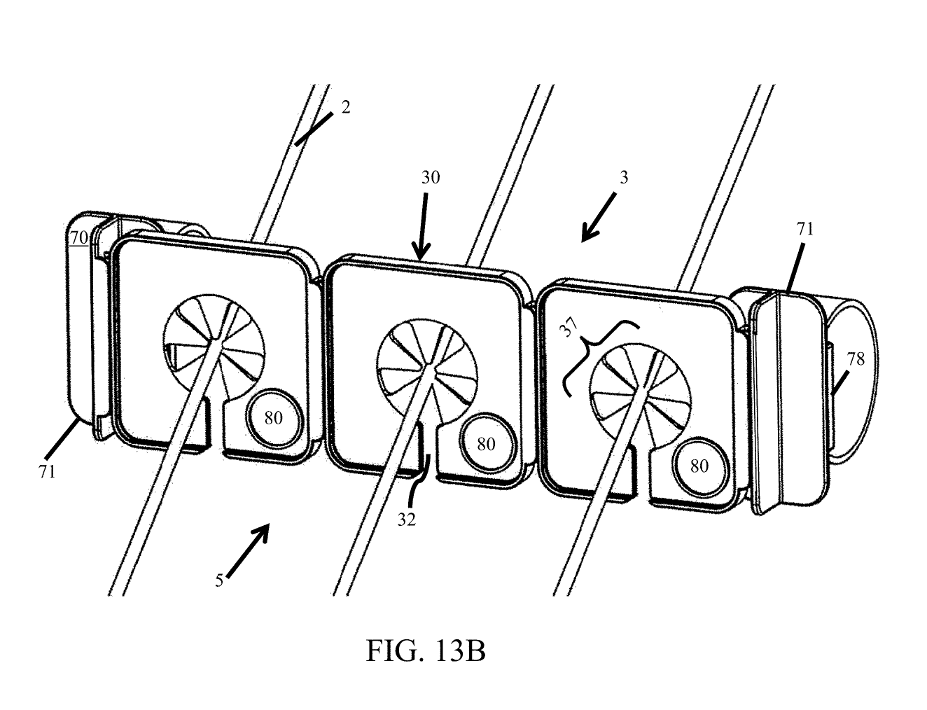

In an alternative embodiment, a guide 30 can have more than one orifice 34, which can further be connected by channels 37, which allow a medical line to be moved from one orifice to another. This embodiment allows a medical line 2 to be pushed through the insertion slot 32 and into an orifice 34, from which one or more secondary orifices 35 can be reached with the channels 36. FIG. 13A illustrates a non-limiting example of a guide having an insertion slot leading to an orifice and multiple secondary orifices connected thereto with channels.

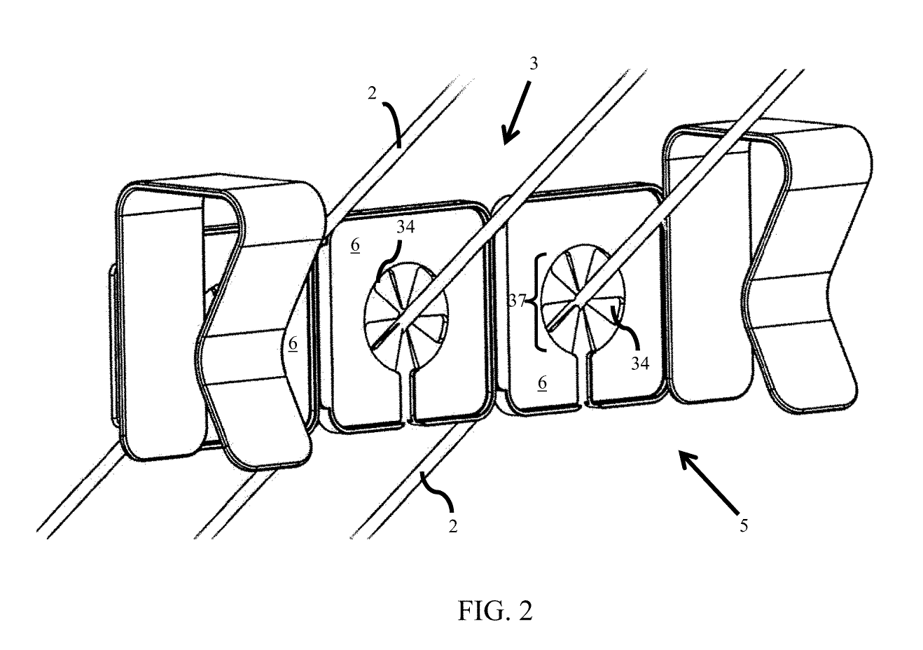

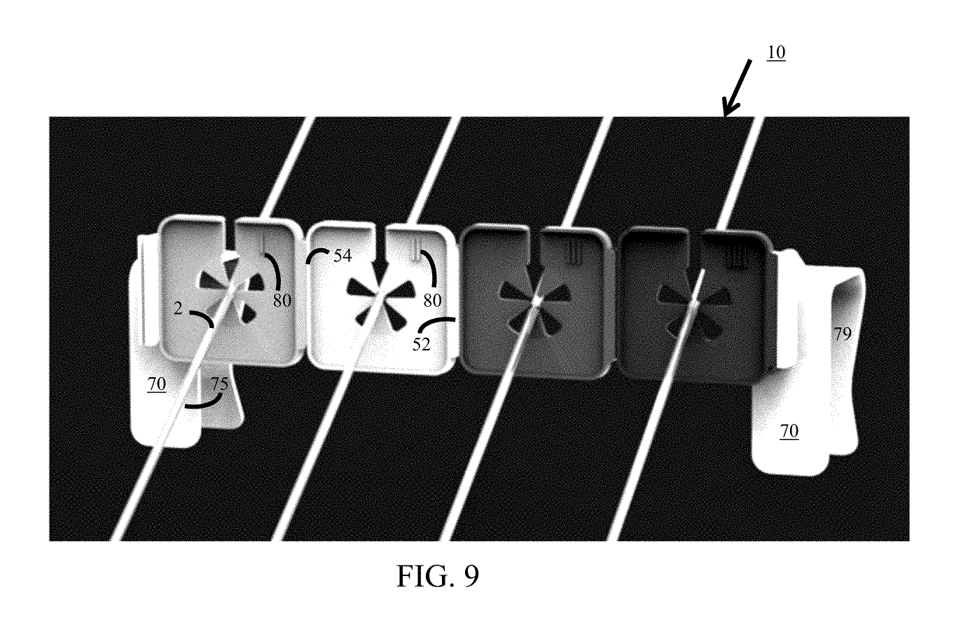

It can be helpful, though not required, for a guide to be positioned so that the insertion slot is directed distally 5, as shown in FIGS. 2, 7 and 14. This can prevent the insertion slot from being a potential “catch” for objects that traverse across it. For example, when attending to a patient, a healthcare provider could lean over, or other objects can be moved over, a medical line organizer 10 attached to a bed rail. If the insertion slot 32 is directed upwards, as shown, for example, in FIG. 9, the clothing or objects can become ensnared in the insertion slot. In some instances, however, such as where the location of a medical line organizer is less likely to be engaged or contacted, the direction of the insertion slot may not matter. Thus, the alternative configuration in FIG. 9, where the guides are arranged with the insertion slots directed proximally 3, can be a suitable alternative embodiment.

The side-to-side width (SSW) of an insertion slot will vary depending upon numerous factors understood by those with skill in the art. In one embodiment, a slot can have a side-to-side width of at least 0.2, 0.3, 0.4, 0.5, 0.6, 0.7, 0.8, 0.9, 0.10, 0.11, 0.12, 0.13, 0.14, 0.15, 0.16, 0.17, 0.18, 0.19, 0.20, 0.21, 0.22, 0.23, 0.24, or 0.25 inches or a side-to-side width in a range between any two of the listed values. In a particular embodiment, the side-to-side width of a slot is between approximately 0.625 inch and approximately 0.1875 inch. In a specific embodiment, the side-to-side width of a slot is approximately 0.125 inch.

In a specific embodiment, the material of a guide is sufficiently flexible that the slot can be separated to increase the side-to-side width. With this embodiment, the size of the slot may not be critical, as the slot can be opened to accommodate a variety of medical lines. In a further specific embodiment, some portion of the guide is bendable. This can allow the insertion slot to be enlarged. For example, one side of the insertion slot can be bent toward the rear face and/or the other side of the insertion slot can be bent toward the front face. The selection of a material for a guide can dictate whether such bending is temporary, where the material has shape memory and returns automatically to an original configuration, or whether the insertion slot maintains a bent configuration until returned to an original configuration.

The shape and dimensions of an orifice can depend upon numerous factors that a person with skill in the art will understand. However, there are a multitude of different types of medical lines 2 that can be used with the embodiments of the subject invention. Each one can have a unique circumferential shape, dimensions, material, rigidity, etc. In one embodiment, the shape of an orifice is substantially circular, which can accommodate a wide variety of medical lines styles. This does not preclude an orifice from having any other circumferential shape, such as, for example, square, triangular, oval, or any other polygonal shape. In order to accommodate different types of medical lines 2, the diameter of an orifice can be of a size large enough to accommodate larger medical lines. In one embodiment, the diameter of an orifice is approximately 1.0 inch to approximately 2.0 inches. In a more specific embodiment, the orifice is approximately 1.5 inches in diameter.

As discussed above, a medical line is moved through an insertion slot 32 to the orifice. While the dimensions of an insertion slot can vary, including the side-to-side width (SSW), as shown in FIG. 4, it can be advantageous for the insertion slot to allow medical lines to move through without being significantly deformed. This can be particularly important with flexible tubing or gas lines where the tubing can be damaged or compromised if constricted, compressed, or stretched during insertion into an orifice. However, as described above, it can also be advantageous for the insertion slot to be directed distally. Therefore, the side-to-side width of an insertion slot must also be sufficiently narrow that a medical line does not drop out of or is not easily dislodged from the orifice.

In one embodiment, a diaphragm 37 is disposed within an orifice 34. A diaphragm can be one or more flexible projections that cover at least part of the orifice and can extend towards the center of an orifice. A diaphragm can act as a support for a medical line 2 placed in the orifice, holding or supporting a medical line within the orifice. Ideally, a diaphragm does not interfere with the operation of the medical line or the ability to actively move a medical line into and out of an orifice through the insertion slot or a channel 36. However, a diaphragm can inhibit the accidental or inadvertent removal of a medical line from the orifice.

In one embodiment, a diaphragm is a flexible sheet 38, such as, for example, a membrane, or panel of material, that extends over an orifice. The flexible sheet can be sufficiently taut within the orifice, so that it holds a medical line in place within the orifice. There can further be a slit in the diaphragm, contiguous with the insertion slot 32, which allows a medical line to be moved towards the center of the orifice. The flexible sheet can stretch, bend or otherwise expand or enlarge, to accommodate a wide range of medical lines diameters and shapes, such that the flexible sheet presses against at least a portion of the medical line with sufficient force to hold it in place within the orifice and not affect operation of the medical line. FIG. 13A illustrates one non-limiting example of a flexible sheet diaphragm 38.

In another embodiment, a diaphragm is formed by one or more projections or fingers 39 that extend into the orifice. The fingers can allow a medical line to come through the insertion slot 32 and pass into the orifice, where one or more of the fingers make contact with the medical line. The fingers can also stretch, bend, or otherwise be deformed so that they can accommodate medical lines of a wide range of diameters and shapes without affecting the operation of the medical line. FIGS. 1, 3A, and 10 illustrate non-limiting examples of multiple fingers 39 forming a diaphragm.

As mentioned above, it can be beneficial for the insertion slot to have a side-to-side width (SSW) that does not damage a medical line pushed or moved therethrough. Thus, in certain embodiments, the side-to-side width of an insertion slot can be only slightly greater than the diameter of a medical line. It can be further advantageous for a guide to be positioned with the insertion slot directed distally to inhibit catching on the edges of the insertion slot. However, this can position the medical line directly against the edge of the orifice and/or the insert slot where it opens into the orifice. The use of a diaphragm within the orifice of a guide can aid in ensuring that the medical line is held away from the insertion slot and can inhibit the medical line from resting against the insert slot. A person skilled in the art will be able to determine any of a variety of diaphragms or similarly-functioning structures that can be used with the embodiments of the subject invention. Such variations, which provide the same functionality, in substantially the same way with substantially the same result, are within the scope of this invention.

To maintain organization of medical lines and increase the safety in a patient environment, the guides can be attachable to a bedside support 100 that holds the guides and any medical lines 2 therein in a stable location or position. For example, the guides can be attached to the bedrails often present on a standard hospital bed or a vertical rolling pole often used to support I.V. bags and other equipment. They can also be attached to equipment or other devices that may be present within a patient environment.

The guides can be used separately, where each guide can be attached to a bedside support. The guides are most amenable for use in situations where more than one medical line is present in the patient environment. Accordingly, it is more likely that multiple guides will be used in a patient environment. Each guide can be separately attachable to a bedside support by numerous techniques known to those with skill in the art. This can include, but is not limited to, such devices and methods as adhesives, magnets, clips, ties, hooks, straps, and other devices known in the art or combinations thereof, which can be attached directly to a guide.

The guides can also be connectable to each other, allowing them to be placed and moved as a single unit, instead of handling each individual guide. As the medical lines in a patient environment are changed, increased, or removed, the number of guides connected together can also be changed accordingly. Guides can be made connectable by any of numerous techniques and devices, known to those with skill in the art. It can be preferable for any connectable structure 40 utilized with a guide to provide easy interchangeability, so that the guides are not damaged, changed or altered.

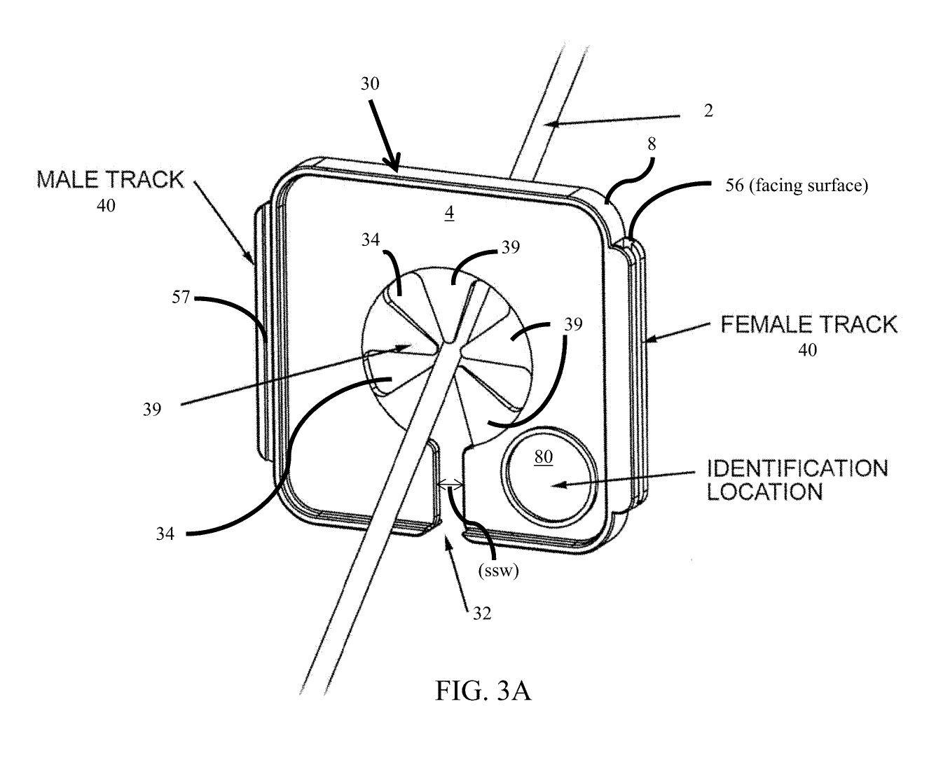

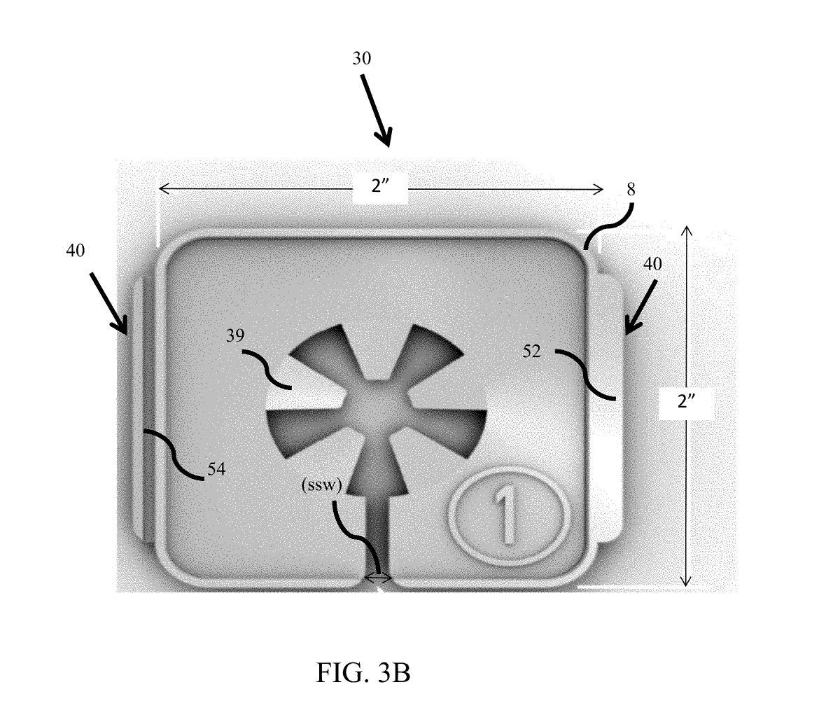

In one embodiment, a connectable structure 40 employs elongated, continuous or intermittent, compatible tracks 50 to connect one or more of the circumferential edges 8 of two or more guides 30. Compatible tracks can have two interdigitating profiles configured to hold the track together when engaged. More specifically, compatible tracks can have a female track 52 on one guide and a male track 54 on another guide that can be snapped, zipped, or slid together, or otherwise joined to removably attach one guide to another guide, which is illustrated, by way of example, in FIGS. 4 and 5. A specific embodiment utilizes an interdigitation of the tracks that causes the tracks to lock or engage with each other in a way that prevents connected guides from coming apart unless slid apart. For example a T-slot, dovetail, or similar type of interdigitation can be used with the track, such that the guides can only be engaged by sliding the tracks over one another and cannot be separated by pulling one track against or across another track or pulling perpendicularly to the tracks. FIGS. 3B, 5, and 10 illustrate one non-limiting example of a male track having a generally T-shaped cross-sectional shape and a female track having a similar T-shaped track for receiving the male track. In this embodiment, a facing surface 56 on the female track is juxtaposed against a shoulder 57 on the male track, an example of which is shown in FIGS. 3A, 4, and 5. Once engaged, the male track cannot be disengaged from the female track except by sliding the male and/or female tracks in opposite directions.

In a further embodiment, illustrated in FIG. 5, each guide can have both a female 52 and a male 54 track to facilitate the guides being interchangeable with each other and arranged in any order. In a specific embodiment, a guide has a female track 52 on one circumferential edge 8 and a male track 54 on another, or another part, of the, circumferential edge. The male and female tracks can be perpendicular and directly opposite from each other on a guide. Alternatively, they can be located elsewhere on the circumferential edge where they are not directly opposite from each other. FIGS. 1, 4, and 9 illustrate one embodiment where the female and male tracks are directly opposite to each other on a guide. With this arrangement, the guides can be connected in a linear or side-by-side fashion, as shown. If the tracks are not oppositely arranged, the guides can be attachable in a non-linear fashion (not shown).

In an alternative embodiment, a connectable structure 40 can be a tab and slot configuration. This embodiment could also employ female 52 and male 54 tracks, which have more of a “tab and slot” configuration. With this embodiment the configuration or shape of the tracks does not specifically hold them together. Instead, friction force or “stiction” between the male and female tracks is used to maintain the connection. For example, the tracks could be made of a material that provides sufficient frictional force to hold the male (tab) 54 track in the female (slot) 54 track. For example, tracks made of rubber, nylon, or certain plastics can exhibit sufficient friction forces between the components to hold them in place. Alternatively, there can be structures on either track that create or increase the frictional force between the tracks. By way of example, one or both of the tracks can have ribs, nibs, rises, or other extended surface features that engage with the opposite track to hold it in place and together. One embodiment discussed below utilizes wings 61 that can increase stiction between tracks.

In a particular embodiment, a connectable structure 40 uses a female 52 and a male 54 track that have rigid or semi-rigid interdigitating profiles. Connecting the tracks requires aligning the ends of compatible tracks and sliding the male track into the female track, which is illustrated in the example in FIG. 4. To prevent or inhibit the tracks from sliding apart uncontrollably or unintentionally, at least one track stop 60 can be incorporated with the guide of one or more of the tracks. A track stop can permit two tracks that have been engaged to slide along their lengths to a certain point or for a certain distance, at which they are stopped from moving or sliding any further.

A track stop can employ or include any of a multitude of devices known to those with skill in the art. A track stop used to inhibit motion between two tracks usually employs structures in or on one or more of the tracks that abut against each to prevent, or at least inhibit, further sliding. One simple iteration known in the art, has one end of a female track blocked with some structure to prevent the male track from sliding further than the end of the female track. Other examples include one or more abutment structures along the length of one track that juxtapose against a structure within the opposite track preventing the tracks from sliding further than allowed by the abutment structure.

In one embodiment, the male track has at least one wing 61 in the form of a proximally 3 angled projection, such that there is formed a proximally facing shoulder 63 on the male track. In a particular embodiment, the wing is located on the arm 55 of a T-shaped male track, as illustrated in FIGS. 6A and 6B. In a further embodiment, the female track has a distally 5 facing shelf 62 at about the proximal end 3 of the compatible T-shaped channel 53, shown, for example, in FIGS. 5 and 6A. With this embodiment, the distal end 5 of the female track can be aligned with the proximal end 3 of the male track and the female track can be slid onto or moved over the male track. As the female track moves distally, the shelf 62 in the female track will abut against the shoulder 63 of the male track, as demonstrated in FIG. 6A. This can stop, or at least inhibit, the distal progress of the female track. This embodiment provides a further advantage of also providing or increasing a friction fit between the male and female tracks, further securing the tracks together until deliberately separated by reversing the above process.

As discussed above, one or more guides can be attached or coupled to a bedside support by numerous techniques known to those with skill in the art. The guides can be attached individually, or they can be joined, as previous described, and coupled to a bedside support 100 as a single unit. Mechanisms and apparatuses that can be used to couple one or more guides to a bedside support include, but are not limited to, adhesives, magnets, clips, ties, hooks, straps, wraps, and other devices known in the art, or combinations thereof. The guides and any coupler mechanism or apparatus can also be separable. This can allow various combinations of guides and coupler 70 devices. Some guides can be attached to a bedside support and other guides, not attached to a bedside support, can, instead, be coupled to only another guide. A coupler 70 device can also be fixedly or removably attached to a guide. For example, a coupler 70 can be permanently connected to the rear face 6 and/or a circumferential edge 8 of a guide. Alternatively, it can be removably attached to already existing features on a guide, such as to tracks, described herein, or guides can be made to include various features for joining to an attachment mechanism. A removable attachment mechanism can be attached to a guide with any type of device or apparatus known that allows a guide and an attachment mechanism to be removably joined. This can include such devices and methods as tab and slot configurations, snap-on features, paired magnetic connectors, clips, and other devices and methods known in the art, or combinations thereof. Attachment mechanisms can also be formed as part of the guide, such that they are not removable. This can include, by way of non-limiting example, clips, ties, clasps, or other devices that remain attached to or integrated into a guide for attaching the guide to a bedside support. A person skilled in the art will be able to determine any of a variety of techniques, devices, or methods that can be used to removably and/or fixedly join an attachment mechanism to a guide. Such variations, which provide the same function, in substantially the same way, with substantially the same result, are within the scope of this invention.

One embodiment utilizes at least one coupler 70 that attaches to a guide, where an attachment mechanism can be either permanently or removably attached to the coupler. A coupler can be attached to a guide by any of the one or more devices or techniques discussed above. In a particular embodiment, a coupler has one or more tracks 50 that are compatible with one or more tracks on a guide, as disclosed above. The location of the tracks on a guide can determine how the compatible tracks on a coupler will be arranged. FIGS. 3A, 4, and 7 illustrate an embodiment where the tracks are located on different areas of the circumferential edge 8 of a guide. More particularly, they are positioned in opposing directions on the circumferential edge of a guide.

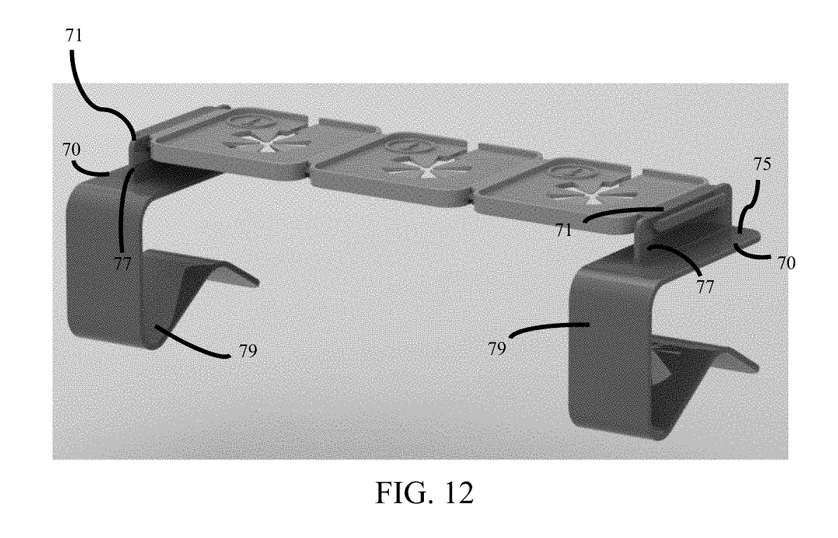

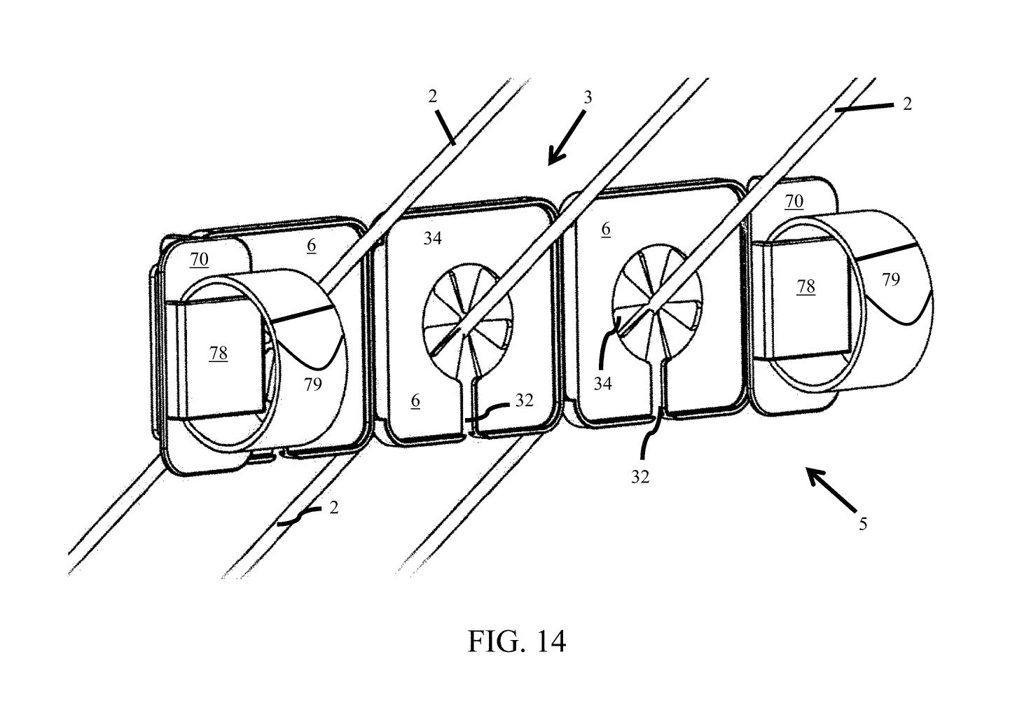

In a specific embodiment, there is a female coupler 72 and a male coupler 73 that are removably attachable to the male tracks 54 and the female tracks 52, respectively, on a guide 30. FIG. 13B illustrates one example of this embodiment. The tracks can be attached to a coupler in any of a variety of locations or configurations. For example, they can be attached directly to the front panel 75 of a guide to be compatible with tracks on associated guides. Alternatively, there can be track support 77, as shown, for example, in FIG. 13, to which a set of tracks can be attached. The track support can extend from the front panel 75 in any direction, such as, for example, generally perpendicularly, as shown. This allows the tracks attached thereto to be at an angle to the front panel 75. As will be detailed below, the coupler can be attached to any of a variety of holders 79 that can be used to attach a coupler to a bedside support 100. For the embodiment above, where the guides have tracks on the circumferential edge, the use of a track support 77 can align a guide 30 with a holder 79 so that they can be attached in a linear fashion, an example of which is shown in FIGS. 9 and 13A. So, for example, multiple guides having male and female tracks can be removably attached together, linearly or otherwise. At either end of the attached guides there will be an unattached set of male tracks on one end and an unattached set of female tracks on the other end. A male or female coupler having a track support with tracks 50 thereon can be attached to a respective end of the series of guides, an example of which is shown in FIG. 12. This can position the coupler planarly with the guide. In a particular embodiment, the coupler will be planar with the rear face 6, as shown in the example in FIG. 14. A holder 79 attached to the coupler can then be directed so that it can be connected to a bedside support, which is demonstrated in the example in FIG. 1.

In a further embodiment, a coupler can be made universal, such that it is configured with both female 52 and male 54 tracks. As described above, each set of tracks can be attached to the front panel 75, or to a track support 77, or some combination thereof. This universal coupler 71 can be advantageous because it can be used with either female or male tracks, which can eliminate the number of different types of pieces or components necessary for utilizing the embodiments of the subject invention. FIGS. 1, 12, and 13A provide non-limiting examples of universal coupler embodiments. With all of these embodiments, one or more couplers can be used with one or more guides. It will be understood that it is possible for a single coupler to be attached to one guide or to a series of connected guides, if it is not necessary, or possible, to support the guide or guides at both ends. If a universal guide 71 is employed, one or more guides can be attached to one or both sets of tracks on the universal guide. This can allow tracks to extend from either or both sides of a coupler, which is not shown in the Figures, but will be understood by a person skilled in the art.

To attach one or more couplers to a bedside support, there can be a holder 79 operatively connected to a coupler. A holder can be any device, structure, or apparatus capable of securing one or more couplers to a bedside support. In one embodiment, a holder is contiguous with a coupler, such that the coupler and holder are formed as one unit. Alternatively, the holder and coupler can be separable, such that the coupler and holder are separate components that can be used together. A person skilled in the art, having benefit of the subject disclosure, would be able to devise or create any of a variety of holder designs and configurations that can be used with the embodiments of the subject invention. Such variations are within the scope of this invention.

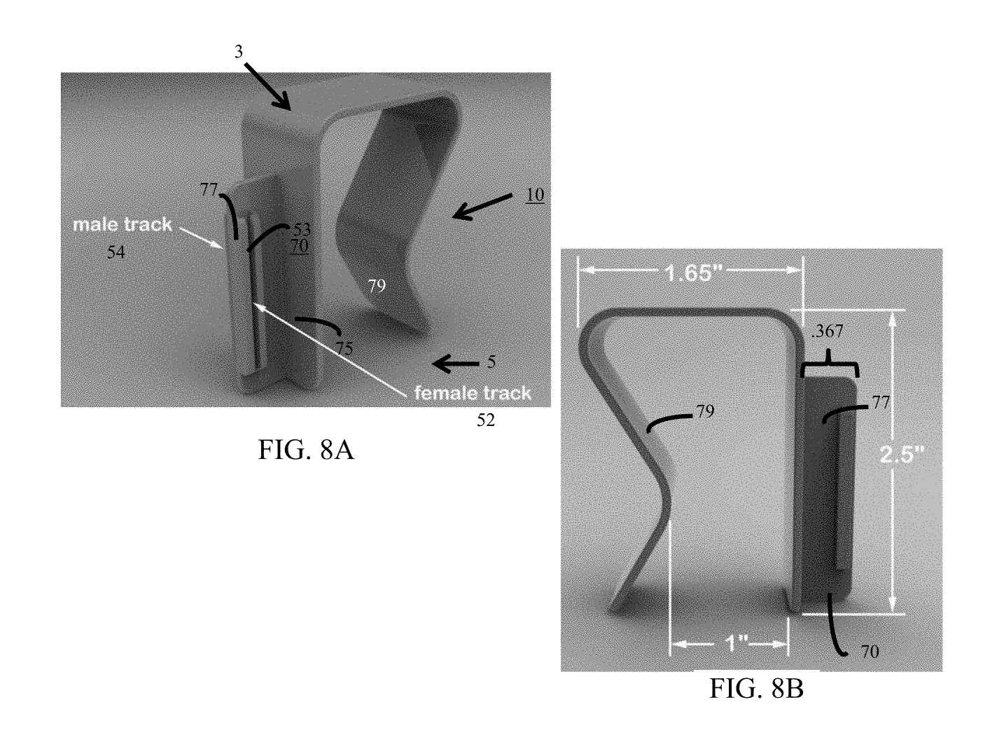

In one embodiment, a holder is configured as a clip fixedly attached to a coupler 70. In a more specific embodiment, the clip is integrated with the structure of a coupler, wherein the coupler forms part of the clip. FIGS. 2 and 12 illustrate one non-limited example of this embodiment. The clip can have any design or configuration that allows it to be attachable to a bedside structure. Such design can be specific for a particular bedside structure or it can be more universal in operation, for attachment to several different types of bedside structures. FIGS. 8A and 8B illustrate a specific embodiment of a clip that can go over a bedside rail.

In another embodiment, a holder is configured as a flexible band that can be attached to a bedside support. The use of a band can allow a coupler to be attachable to more types of bedside supports. A band can be fixedly attached to a coupler, such as with rivets, screws, adhesives, heat or pressure sealing, and with other devices and techniques known in the art. Alternatively, the coupler can be configured with structures to which a band can be removably attached. One embodiment utilizes a sleeve 78 fixedly attached to the back of a coupler through which a band can be disposed, such as shown, in the examples in FIGS. 13B and 14. The ends of the band can be connectable with snaps, hook-and-loop material, formable wires, and other devices and techniques known to those with skill in the art.

Different types of indicia 80 are often utilized for identification of specific medical devices and equipment. Such practice can aid in appropriate identification of devices, which can increase patient safety. The guides 30 and couplers 70 of the subject invention can likewise include one or more indicia to identify or at least distinguish between different types of medical lines used therewith. Indicia can include such features and techniques as color-coding, alphanumeric symbols, guides of different shapes, and other techniques or combinations thereof known to those with skill in the art. FIG. 9 illustrates an example where each guide has a different color to better identify each of the medical lines therein. FIG. 9 also illustrates how guides can have more than one type of indicium 80, in this case both color-coding and symbols are used to distinguish between different guides.

As mentioned above, medical lines 2 can drape all through a patient environment and cross-over or cross-under each other and other equipment. The ability to identify a medical line going to or from a patient is critical. Likewise, the ability to identify the direction or path of medical lines in a patient environment can also be critical. While a healthcare provider can physically follow the path of a line to or from a patient and/or medical device, such method can be time-consuming and can still be prone to errors.

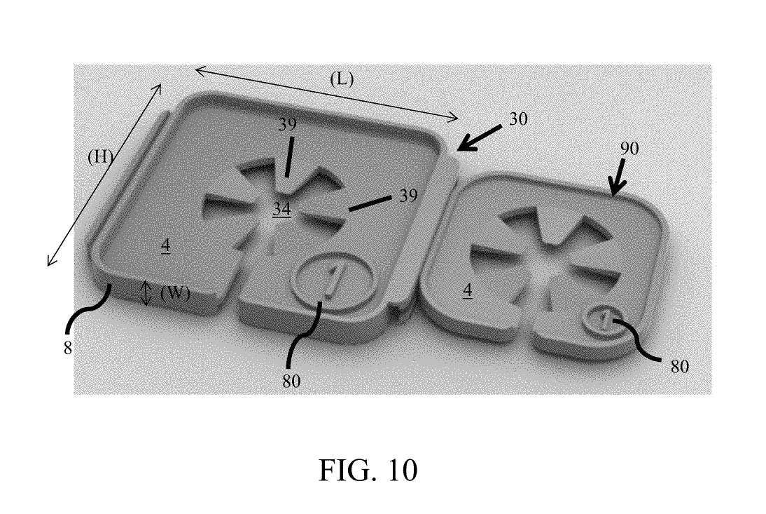

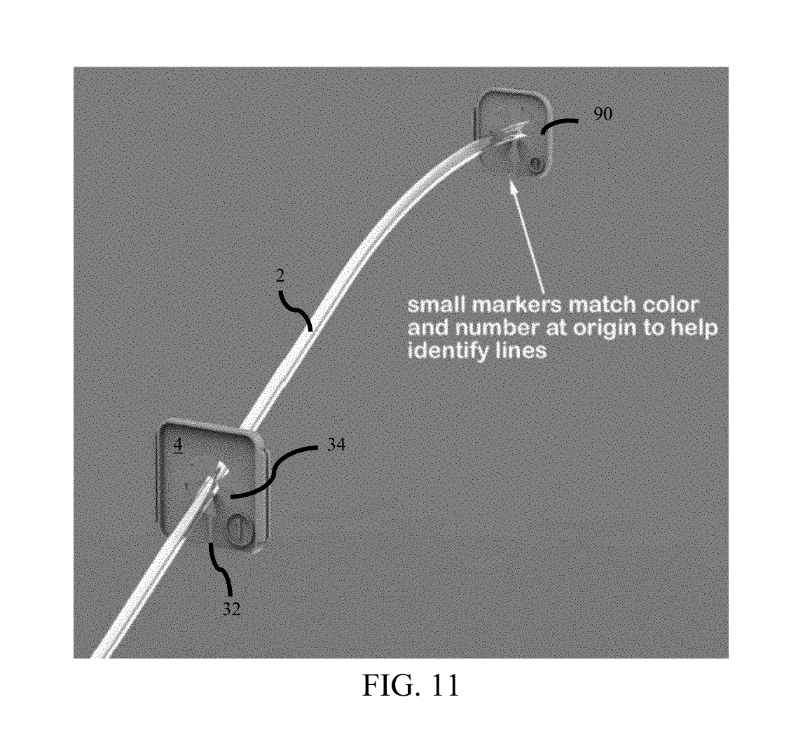

The embodiments of the subject invention can include the use of at least one marker 90 that can be removably attached along the length of a medical line. A marker can have indicia that are identical to or at least similar to those used with a guide, whereby the guide and one or more markers can be identified as being similar. A medical line secured in a guide 30 can also have one or more markers with similar or identical indicia thereon attached along the length of the medical line. A healthcare provider can then identify a medical line by the associate guide and then follow the course or path of the line more easily by finding the one or more markers attached thereto. FIG. 10 illustrates one embodiment of a marker 90 having indicia (color, alphanumeric symbol, and shape) that are similar to an associated guide 30. FIG. 11 illustrates an example of how a medical line can be secured to a guide 30 and the marker can be placed elsewhere along the length of the medical line to aid in identifying the line in a patient environment.

In a further embodiment, a marker 90 can be removably attachable to a guide. This can make the matching sets of guides and markers easy to store, and can also allow them to be kept together in the patient environment until ready for use. Markers and guides can be connectable in a variety of ways. In a specific embodiment, the markers can have one or more tracks 50 that can be connected to a guide that has one or more compatible tracks. FIG. 10 further illustrates an embodiment wherein a guide has a track to which a marker has a compatible track thereon that can be attached to a track on the guide. This example shows the marker with a single track. However, additional tracks, male and/or female, can be present on a marker, such that it can be attached to more than one type of track on the guide and more than one marker can be attached to a guide or to another marker.

In a specific embodiment, a marker is, in general, a smaller version of a guide and can be used in similar fashion to a guide. Thus, the factors and embodiments discussed above with regard to guides 30 can be equally applicable to and are reiterated here with regard to markers and their use.

The ability to identify medical lines in a patient environment can be a critical aspect of patient care and safety. The embodiments of the subject invention provide quick and easy to use devices and methods for identifying one or more medical lines in a patient environment. The medical line organizer described herein can be employed with already existing medical devices and bedside structures and do not inhibit the use of medical lines in anyway. Additional embodiments can enhance medical line identification by providing markers that correspond to particular guides on the medical line organizer and attach along the length of a medical line to aid in identification of a medical line along its full length. The embodiments described herein represent a significant improvement in overall patient care and can significantly reduce healthcare provider errors and hospital costs.

The scope of the invention is not limited by the specific examples and suggested procedures and uses related herein since modifications can be made within such scope from the information provided by this specification to those skilled in the art.

All patents, patent applications, provisional applications, and other publications referred to or cited herein are incorporated by reference in their entirety, including all figures and tables, to the extent they are not inconsistent with the explicit teachings of this specification. Additionally, the entire contents of the references cited within the references cited herein are also entirely incorporated by reference.

The examples and embodiments described herein are for illustrative purposes only and that various modifications or changes in light thereof will be suggested to persons skilled in the art and are to be included within the spirit and purview of this application.

The invention has been described herein in considerable detail, in order to comply with the Patent Statutes and to provide those skilled in the art with information needed to apply the novel principles, and to construct and use such specialized components as are required. However, the invention can be carried out by specifically different equipment and devices, and various modifications, both as to equipment details and operating procedures, can be effected without departing from the scope of the invention itself. Further, although the present invention has been described with reference to specific details of certain embodiments thereof and by examples disclosed herein, it is not intended that such details should be regarded as limitations upon the scope of the invention except as and to the extent that they are included in the accompanying claims.

What is claimed is:

1. A device for organizing medical lines, the device comprising:
at least one guide having:
a circumferential edge;
a front face;
a rear face;
an orifice that communicates the front face with the rear face;
an insertion slot that extends from the orifice, through the guide and opens onto the circumferential edge;
at least one connectable structure on the at least one guide by which two or more of the at least one guide can be removably connected; and
at least one coupler operably connectable to one or more of the at least one guide for attaching the at least one guide to a bedside structure.

2. The device for organizing medical lines, according to claim 1, further comprising;
one or more indicia for distinguishing between two or more of the one or more guides.

3. The device for organizing medical lines, according to claim 2, wherein the connectable structure comprises at least one set of tracks.

4. The device for organizing medical lines, according to claim 3, wherein the tracks on each guide are compatibly interdigitated male tracks and female tracks.

5. The device for organizing medical lines, according to claim 4, wherein each at least one guide has one set of male tracks and one set of female tracks.

6. The device for organizing medical lines, according to claim 5, further comprising tracks on the coupler, such that the coupler can be operably connected to tracks on the at least one guide by sliding the tracks over one another.

7. The device for organizing medical lines, according to claim 6, wherein a coupler comprises one set of male tracks and one set of female track, such that the coupler can be connected to the male tracks or female tracks on a guide.

8. The device for organizing medical lines, according to claim 7, further comprising one or more markers comprising:
a circumferential edge;
a front face;
a rear face;
an orifice that communicates the front face with the rear face;
an insertion slot that extends from the orifice, through the guide and opens onto the circumferential edge; and
at least one connectable structure on the at least one marker by which the one or more markers can be removably connected to a guide.

9. The device for organizing medical lines, according to claim 8, further comprising a holder operatively connected to the at least one coupler.

10. The device for organizing medical lines, according to claim 9, further comprising a track stop.

11. The device for organizing medical lines, according to claim 10, wherein a track stop comprises a wing having a shoulder on the male track and a shelf in the female track, whereby the juxtaposition of the shoulder and the shelf stops sliding of the tracks.
